(19) United States
(12) Patent Application Publication
(10) Pub. No.: US 2023/0398020 A1
(43) Pub. Date: Dec. 14, 2023

(54) OPHTHALMIC RETINA CONCURRENT SEALANT MIXING AND ILLUMINATED ASSEMBLY AND METHOD

(71) Applicant: Alcon Inc., Fribourg (CH)

(72) Inventors: Paul R. Hallen, Colleyville, TX (US); William F. Ryann, San Antonio, TX (US)

(21) Appl. No.: 18/321,127

(22) Filed: May 22, 2023

Related U.S. Application Data
(60) Provisional application No. 63/366,014, filed on Jun. 8, 2022.

(51) Int. Cl. A61F 9/00; A61M 5/19

(52) U.S. Cl. CPC A61F 9/0017; A61M 5/19; A61B 1/07

(57) ABSTRACT

A syringe with multiple chambers of isolated constituents for advancing to a mixing chamber in furtherance of delivery to tissue within an eye of a patient. The syringe also includes a mixing mechanism that is in communication with the mixing chamber. Additionally, the isolated chambers may be one of sequential or side by side/parallel depending on the types of constituents to be combined and other surgical preferences.

### BACKGROUND

Over the years, many dramatic advancements in the field of eye surgery have taken place. One of the more common eye surgery procedures is a vitrectomy. Vitrectomy is the removal of some or all of the vitreous humor from a patient's eye. In some cases, where the surgery is limited to removal of clouded vitreous humor, the vitrectomy may constitute the majority of the procedure. However, a vitrectomy may accompany cataract surgery, surgery to repair a retina, to address a macular pucker or a host of other issues.

Often a vitrectomy is accompanied by a variety of other procedures to address more specific eye features. That is, in addition to the described vitrectomy, other types of probes or implements may be utilized to address specific eye issues. The scenario may involve a degree of vitrectomy followed by the use of an implement to directly interact with an eye feature. For example, repair to retinal tissue at the back of the eye may be undertaken by various forceps, scissors or other implements that are utilized following some degree of vitrectomy. A vitrectomy may also follow retinal tissue repair to remove loose debris or stray tissue.

Following retinal tissue repair and any accompanying vitrectomy procedures, efforts are often undertaken to shield and protect the repaired retinal tissue to allow for a period of isolated healing. One technique for shielding and/or protecting the repaired retinal tissue is to follow the repair with the introduction of a silicon oil. This viscous oil may serve to isolate the retina and allow for a fairly extended period of shielded healing. For example, the oil may be left in the eye for a period of 90 days or perhaps longer.

Alternative efforts to protect and shield the repaired retina have been developed that may avoid the need for a subsequent removal surgery. For example, a gas tamponade of sulfur hexafluoride or other dilute medical gases may serve to facilitate healing of a repaired retina. Utilizing a gas tamponade as an aid in healing over the long term may require the patient to actively participate in the healing process by spending some time face down to position the tamponade bubble at a retinal healing location of the eye.

Another effort to present a wound isolating technique to a repaired retina while avoiding a subsequent removal surgery involves the placement of a retina “patch” over the retina. In this way, a more discrete placement of a degradable substance is utilized to achieve the isolation during the healing period. Due to the degradable nature of the substance, follow on removal surgery may not be required.

The “substance” which constitutes a retinal patch may include a mixture of different constituent components that, upon mixing, may congeal and set in a very short period of time. For example, a solid polyethylene powder mix combined with a liquid polyethylene mix may generally set within about 5 minutes. This means that the surgeon or surgical assistant is presented with separate mixtures that must be combined, mixed and delivered to the surgical site at the back of the eye within a matter of minutes. Otherwise, the patch material may become stuck within the delivery needle or delivered in a clumpy undesirable fashion that may impact the effectiveness of patch performance. Indeed, rather than risk the latter, it is quite common for prematurely mixed patch material to become lodged within the delivery needle or tool, inefficiently necessitating multiple mixture attempts and scrapped delivery tools before proper patch delivery is realized.

### SUMMARY

A material delivery assembly is disclosed for eye surgery. The assembly may be a syringe and includes separate chambers for housing separate isolated constituents. The constituents may be mixed together in one of the chambers or within a third separate mixing chamber. A needle in fluid communication with the chamber within which the constituents are mixed is included with the assembly for positioning adjacent a tissue site in the eye to deliver the mixed constituents thereto.

### BRIEF DESCRIPTION OF THE DRAWINGS

FIG. 1 is a side perspective view of an embodiment of a multi-chamber isolated constituent syringe for eye surgery.

FIG. 2A is an enlarged view of a cross-section of a plunger of the syringe of FIG. 1 advancing a constituent past a membrane barrier of a first chamber and into a second.

FIG. 2B is an enlarged view of a cross-section of the syringe of FIG. 1 wherein a mixing mechanism is employed to initiate mixing of the constituents.

FIG. 2C is an enlarged view of a cross-section of the syringe of FIG. 1 wherein the constituents are mixed within a mixing chamber and advanced through a filter to a needle.

FIG. 3 is an enlarged and partially cross-sectional view of the needle of the syringe of FIGS. 1 and 2C reaching toward a retinal surface within an eye for delivery of the constituent mixture.

FIG. 4 is an alternate embodiment of a multi-chamber isolated constituent syringe for eye surgery.

FIG. 5 is a flow-chart summarizing an embodiment of utilizing a multi-chamber isolated constituent syringe for eye surgery.

### DETAILED DESCRIPTION

In the following description, numerous details are set forth to provide an understanding of the present disclosure. However, it will be understood by those skilled in the art that the embodiments described may be practiced without these particular details. Further, numerous variations or modifications may be employed which remain contemplated by the embodiments as specifically described.

Embodiments are described with reference to certain types of micro-invasive vitreoretinal procedures carried out with a unique assembly or syringe. In particular, a procedure in which a syringe is used to mix and deliver a retinal patch mixture is illustrated and detailed. Of course, a variety of other procedures may be carried out with the types of assembly or syringe embodiments detailed herein. Regardless, so long as the syringe is outfitted with isolated constituent chambers and at least one enhanced mixing mechanism, appreciable benefit may be realized.

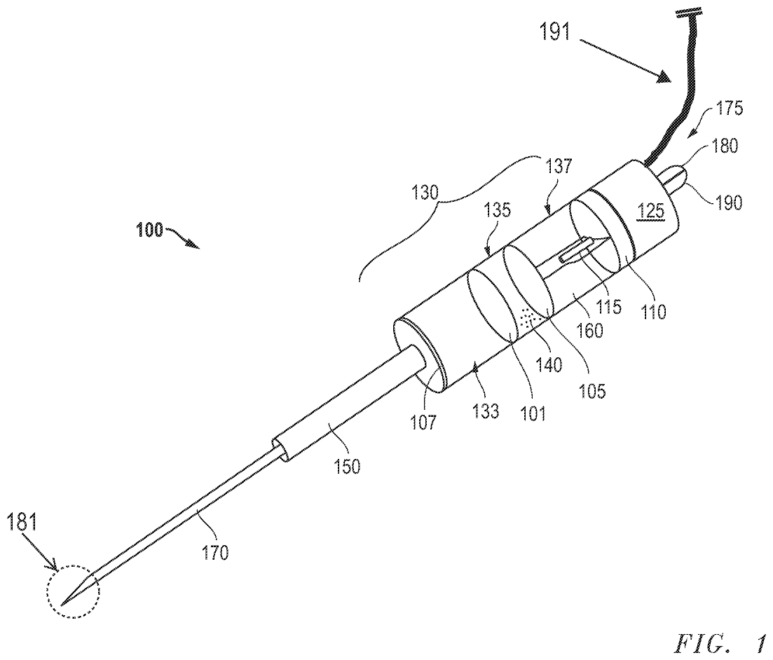

Referring now to FIG. 1, a side perspective view of an embodiment of a multi-chamber isolated constituent syringe 100 is illustrated for eye surgery. The syringe 100 includes a housing 130 with multiple chambers 133, 135, 137. For the embodiment shown, two of the chambers 135, 137 are provided for housing separate constituents 140, 160 in an isolated fashion from one another. Specifically, a substantially dry constituent 140 is housed within a dry chamber 135 and a substantially liquid constituent 160 is housed within a wet chamber 137. Another mixing chamber 133 is provided into which the constituents 140, 160 may eventually be deposited for mixing as detailed further below. However, in other embodiments, use of this separate chamber 133 may be avoided.

Each of the chambers 133, 135, 137 is separated from the others by a barrier in the form of a conventional pressure degradable membrane 101, 105. The membranes 101, 105 may be of a medical foil or conventional rupture disks that are selected to burst upon exposure to a predetermined pressure. For example, in the embodiment illustrated, the syringe 100 may be a 5 milliliter (ml) syringe with a proximal membrane 105 subject to about 100 pounds per square inch (PSI) from a plunger 110 as described below. This degree of pressure may be more than sufficient for rupturing the membrane 105 and facilitating the movement of the liquid constituent 160 from the wet chamber 137 to begin reaching into the dry chamber 135.

The above-described rupture and movement of the liquid constituent 160 begins a process of mixing the liquid constituent 160 with the dry constituent 140 in the dry chamber 135. This may be enhanced by a mixing mechanism 115. In the embodiment shown, this mechanism 115 is an auger/propeller-type of implement that may rotate about an axis through the center of the syringe 100 when directed by an actuator 175. Indeed, the actuator 175 depicted includes two separate depressors 180, 190 wherein one depressor 180 may be utilized to trigger the described plunger 110 movement and the other 190 may trigger the mixing mechanism 115 as noted. In the embodiment shown, these depressors 180, 190 are located together as the actuator 175 given the likely possibility that the surgeon would seek to begin mixing in conjunction with movement of the plunger 110. However, this is not required and the surgeon may seek to begin mixing after plunger 110 movement. The architecture of the actuator 175 would allow for this possibility. Alternatively, the mechanism 115, auger-type or otherwise, may be fixed and non-rotational such that distal advancement alone may generate a swirling type of mixing enhancement.

Continuing with reference to FIG. 1, the described plunger 110 movement and rotation of the mixing mechanism 115 are forcibly facilitated by a pneumatic power cell 125 with locally created pneumatic pressure. In some embodiments, tubing 191 may provide pneumatic pressure (e.g., pressurized air/fluid from a connected console or other pneumatic source) to move plunger 110. In this way, a degree of precision is introduced to the injection process that is not dependent upon the surgeon's thumb or finger positioning at an extension from the plunger. Such pneumatic actuation may also play a role in enhancing mixing through the mixing mechanism 115 illustrated or through other types of mixing enhancing features as detailed further below. Of course, in other embodiments this type of more conventional plunger architecture may also be employed. Additionally, in one embodiment, the mixing mechanism 115 may be telescoping in nature and maintain a physical link to the associated depressor 190. Thus, even after the plunger 110 movement is completed as described below, the surgeon may continue to repeatedly push and release on the depressor 190 to continue rotating the mechanism 115 for mixing the constituents 140, 160. This may be aided by a spring or other conventional return device associated with the depressor 190.

For the embodiment shown, the dry chamber 140 is defined by another, distal, membrane 101. This is not required. However, it may be desirable to include another mixing chamber 133 with dedicated airspace. In this way, after sufficient plunger advancement and pressure buildup, the rupture of the distal membrane 101 will leave a predetermined amount of mixing chamber airspace. This may be tailored to the amount and type of constituents 140, 160 to be mixed. For example, a sufficient amount of inert air or argon gas may be utilized to allow the surgeon to effectively shake the syringe 100 to encourage additional mixing. Further, this may be done in a manner that keeps the dry constituent 140 within an isolated area of the dry chamber 140 without prematurely exposing the constituent to a filter 107 described below or a much larger surface area of the inner wall of the housing 130.

Once mixed, the combined mixture may be forced through a filter 107 at the front of the housing 130 and into a needle 170. The filter 107 may be utilized to prevent any membrane debris from reaching the interior of the needle. In the embodiment shown, a sleeve 150 is provided about the needle 170 which may serve to provide structural support as described further below. While tip 181 of needle 170 is shown as a sharp point, other tip structures could also be used. For example, the tip 181 may include a soft silicone tip, a brush applicator, or a blunt tip (e.g., a stainless-steel blunt tip). The tip structure 112 may aid in applying the mixture to the injection site. For example, in some embodiments, the tip 181 may include a brush applicator that may act to “paint” the combined mixture onto a retina tear. In some embodiments, the combined mixture may be applied in a bead structure along the retina tear to seal the tear. The seal may thus prevent fluid (which could otherwise detach the retina) from getting behind the retina.

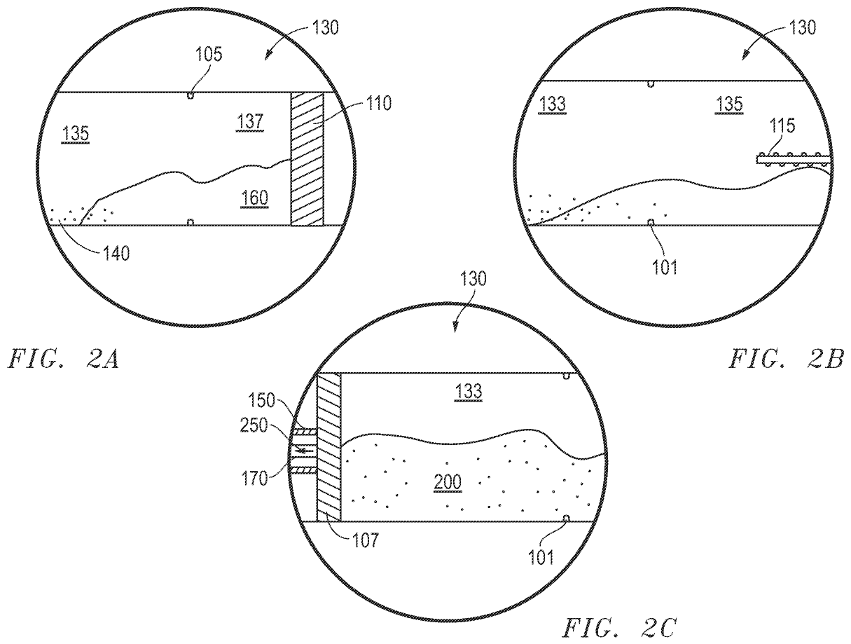

Referring now to FIG. 2A, an enlarged view of a cross-section of the plunger 110 of the syringe 100 of FIG. 1 is shown advancing a constituent 160 past a membrane 105 barrier of a first liquid chamber 137 and into a second dry chamber 135. Only remnants of the membrane dividing the chambers 135, 137 remain and the constituents 140, 160 begin to combine. In one embodiment, the combined mixture of constituents 140, 160 will make up a fast-drying retinal patch. Thus, the syringe 100 is a good tool for quick mixing and delivering of the patch mixture. In one such embodiment, the liquid constituent 160 is less than about 2.0 ml of a polymer solution whereas the dry constituent 140 is another dry polymer reactant that is less than 0.10 ml. Nevertheless, within less than about 5 minutes of exposure to one another, the mixture may become substantially solid.

With specific reference to FIG. 2B, an enlarged view of a cross-section of the syringe 100 of FIG. 1 is shown wherein the mixing mechanism 115 is now illustrated emerging from the plunger 110 of FIG. 2A. The mechanism 115 is employed to more aggressively initiate mixing of the constituents 140, 160 of FIG. 2A as described above. Furthermore, the mechanism 115 may play a role in rupturing the referenced membrane 105. That is, rather than relying solely on pressure, the mechanism 115 may physically breach the membrane 105.

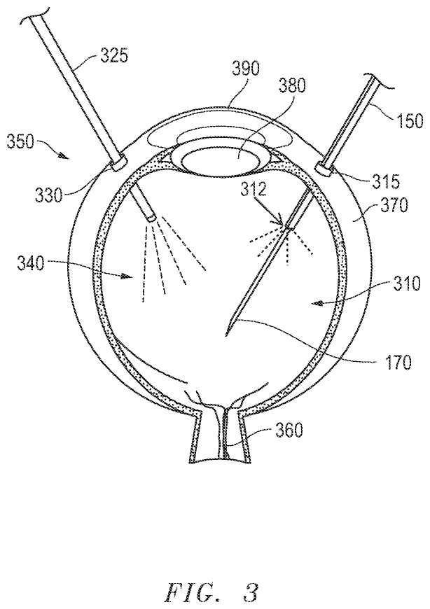

For sake of illustration, the syringe 100 of FIG. 1 and FIGS. 2A-2C is shown in a horizontal manner with a significant amount of airspace throughout the housing 130. However, it is worth noting that, as with other syringe applications, it is likely that the airspace may be less than the substantial amount illustrated and the syringe 100 and housing 130 would be held by the surgeon in a more vertical orientation. In this way, air within each chamber 133, 135, 137 may rise before mixing and delivering the mixture as shown in FIG. 3. Additionally, where the mixing mechanism 115 is of an auger variety, the auger features may extend further laterally toward the sidewalls of any given chamber 133, 135, 137 such that the mechanism 115 leaves substantially minimal amount of clearance, if any. Thus, mixing is further enhanced. Additionally, the mechanism 115 may play more of a role in advancing the mixture distally.

Referring now to FIG. 2C, an enlarged view of a cross-section of the syringe 100 of FIG. 1 is illustrated wherein the constituents 140, 160 are mixed within a mixing chamber 133 and advanced through a filter 107 to a needle 170. Indeed, a flow 250 of the thoroughly combined mixture 200 is illustrated advancing through the needle 170. It is also worth noting that all of the mixture 200 is filtered through the filter 107 before advancing to the needle 170. The primary purpose of the filtering may be to prevent any debris from the ruptured membranes 101, 105 from reaching the needle 170. However, the filter 107 may also be tailored to prohibit any clumped or larger, mostly dry constituent 140 material from reaching the needle 170 which has failed to fully or more properly mix with the rest of the mixture 200 (referred to herein as unmixed constituent). An optional sleeve 150 is also shown about the needle 170 as a structural aid for a surgical procedure which is described further below.

Referring now to FIG. 3, an enlarged and partially cross-sectional view of the needle 170 of the syringe 100 of FIGS. 1 and 2C is illustrated during a micro-invasive vitreoretinal surgery reaching toward a retinal surface above an optic nerve 360 within an eye 350. Thus, the mixture 200 of FIG. 2C may be delivered. Recall that the mixture 200 may be a fast-drying retinal patch that solidifies perhaps within about five minutes of combining as described above. However, as also described above, by simply actuating the syringe 100 of FIG. 1, the mixture 200 may be attained in a matter of moments without any undue concern over premature solidifying.

Continuing with reference to FIG. 3, the needle 170 shown may be 25 gauge or smaller and supported by the sleeve 150 which is guidingly stabilized by a cannula 315 as it reaches into the interior 310 of the eye 350. Another preplaced cannula 330 is also present to guidingly support the introduction of a focused task light instrument 325. In some embodiments, the sleeve 150 may further include a fiber optic for illumination 312. This illumination may be in addition to task light 325 or may be the sole illumination used to deliver the injection (e.g., in the absence of a separate task light instrument 325). While fiber optic illumination 312 is shown terminating on the sleeve, in some embodiments, the fiber optic may further run down the needle to provide illumination closer to the injection site. In some embodiments, the light may be provided approximately 8 millimeters away from the injection site. Other distances are also contemplated (e.g., 1 millimeter, 10 millimeters, etc.) The cannulas 315, 330 may both be retractable to facilitate entry through a valve thereof. Additionally, the cannulas 315, 330 are located at offset positions of scleral tissue 370 to avoid contact with more sensitive eye 350 features such as the cornea 390 or lens 380. Indeed, the surgeon may view the interior 310 of the eye 350 during the procedure directly through the cornea 390 and lens 380, aided by light 340 from the instrument 325. Although, more indirect viewing options may be employed. For example, a wide-angle viewing system with microscope may be utilized.

Given the crowded spacing, the noted wide angle viewing system may be employed along with relatively flexible and long, large gauge tubing. Additionally, the cannula 315 may be articulating. In this way, up to 360 degrees of the retina may be both visible and ergonomically accessible. In one embodiment, the end of the needle 170 includes a fiber optic visual enhancement to further aid in viewing, particularly as the needle 170 comes closer to the retina.

Continuing with focus on accessibility and with added reference to FIG. 2C, in one embodiment, the needle 170 is of a curved shape to facilitate a more ergonomic delivery of the mixture 200. For example, in one embodiment, the needle 170 may be constructed of a memory shape nickel titanium alloy such as nitinol. In this manner, the needle 170 may be straightened for passage through the cannula 315, either by the cannula 315 directly or by temporary retention within the sleeve 150 during the passage. In either case, the needle 170 may return to curved shape form once reaching the interior 310 of the eye 350.

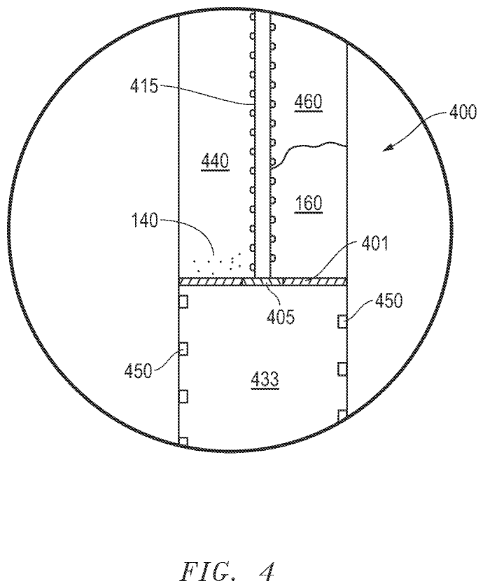

Referring now to FIG. 4, an alternate embodiment of a multi-chamber isolated constituent syringe 400 is illustrated for eye surgery. In this illustration, the syringe 400 is effectively the same as the embodiment of FIG. 1 in terms isolating separate constituents 140, 160 prior to mixing them within a mixing chamber 433. However, the syringe 400 is now more of a double barrel construction with separate chambers 440, 460 arranged such that they would be opened to the mixing chamber 433 at substantially the same time upon breaking of the membrane 401. In this way, the route of successive introduction of liquid 160 to dry 140 component and then both into the mixing chamber 433 may be avoided. This may be beneficial or promote a more evenly distributed final mixture, depending on the nature of the constituents 140, 160.

In addition to the different chamber architecture, the syringe 400 of FIG. 4 includes additional mix enhancing features. For example, the mixing mechanism 415 illustrated operates the same as that of the embodiment of FIGS. 1 and 2B (e.g., see 115). However, in this instance, the mechanism 415 may be longer to provide a more substantial mixing element. Further, the mechanism 415 itself is shown contacting the membrane 410 which defines the mixing chamber 433. Thus, from the moment of advancement of the plunger 110 physical rupturing of the membrane 401 may begin. Indeed, in one embodiment, a separation element 405 may be tailored in size to allow passage of the mechanism 405 by physically dislodging. In one such embodiment, the remainder of the membrane 401 may remain structurally sound as a solid wall barrier to minimize the amount of membrane debris that may potentially require filtering. It is of note that for the embodiment shown, the mixing mechanism 415 would be moving between barrier walls of the chambers 440, 460 that are not illustrated so as to focus on the mechanism 415. Further, for an embodiment utilizing a separation element 405, these barrier walls would be sealingly coupled to the element 405 in advance of its dislodging or separation.

With the constituents 140, 160 introduced to the mixing chamber 433, the longer mechanism 415 may be from about half the length of the chamber 433 up to the full length or even longer. Thus, repeated reciprocation as detailed above may have a substantially greater effectiveness in creating a more homogeneous mixture of the combined constituents 140, 160 within the chamber 433. Indeed, in the embodiment shown, baffling 450 is included which may further enhance the mixing during this reciprocation. That is, a unique combination of stationary baffling 450 may be utilized in conjunction with a moving and reciprocating mixing mechanism 415 to a significantly beneficial effect.

In some embodiments, the mixing chamber 433 may be located adjacent the tip 181, and, in some embodiments, the mixing chamber 433 may be small enough to safely enter the eye when the tip 181 is inserted into the eye. In this embodiment, the constituents 140, 160 are mixed inside the eye in the mixing chamber 433 just prior to entering the eye. For example, the constituents 140, 160 may be delivered along separate channels to the mixing chamber 433 in the eye near the tip 181 and mixed just prior to entry into the eye. The mechanism 115 (e.g., an auger/propeller-type mechanism) may thus spin near the tip 181 such that the constituents 140, 160 mix at the tip 181 just prior to entering the eye. In some embodiments, the constituents 140, 160 may further pass through baffles 450 prior to exiting the tip for additional mixing.

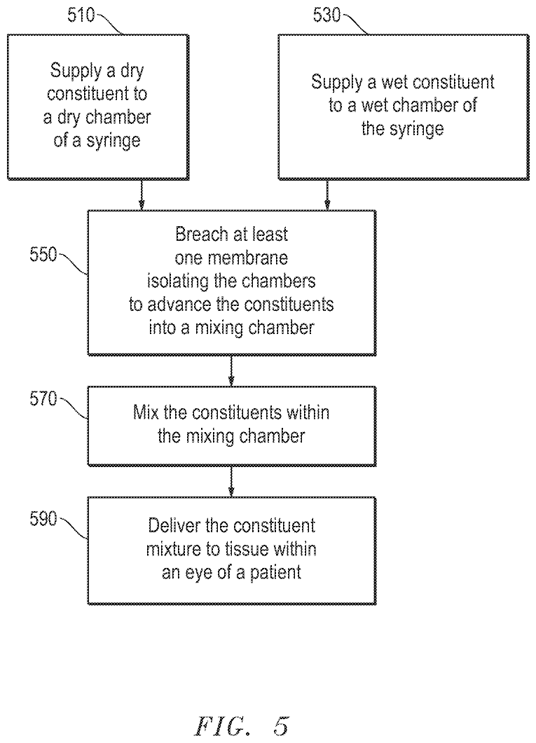

Referring now to FIG. 5, a flow-chart summarizing an embodiment of utilizing a multi-chamber isolated constituent syringe for eye surgery is shown. Namely, dry and wet constituents are supplied to isolated chambers of a syringe as indicated at 510 and 530. At least one barrier or membrane is then breached as noted at 550 to advance the constituents into a mixing chamber where they are mixed (see 570). Thus, as indicated at 590, they may be delivered as a combined mixture to tissue within the eye of a patient.

Embodiments described hereinabove include a device and techniques that allow for an effective manner to attain reliable mixing of constituents for a relatively fast drying combination of components in a very short period of time. This is achieved through a single syringe in an ergonomically preferred manner that provides both mixing and efficiency enhancements.

The preceding description has been presented with reference to presently preferred embodiments. However, other embodiments and/or features of the embodiments disclosed but not detailed hereinabove may be employed. For example, a variety of additional mixing enhancements may be provided within the mixing chamber. This may include the use of a piezoelectric actuator, a spiraling or more circuitous pathway fluidly coupled between the chamber and the needle or a host of other architectural features. In an embodiment employing a piezoelectric actuator, the actuator may be discretely disposed within the mixing chamber or incorporated into a mixing mechanism associated with the plunger or any other part of the syringe such that vibrational effects are translated more throughout a given chamber or even the entire syringe. In some embodiments, the ultrasonic crystal oscillation driven mixing elements (e.g., piezoelectric driven elements) may be vibrated using existing ultrasonic driver electronics from a connected console. For example, the same ultrasonic driver electronics that drive piezoelectric handpieces for lens removal could also be used to vibrate the mixing elements (such as metallic agitators) in the syringe. Additionally, the supplied vibration/oscillation could vibrate the syringe barrel for further mixing of the contents. In some embodiments, the pneumatically driven stopper may drive the mixture through the ultrasonic crystal oscillation driven mixing elements. Furthermore, persons skilled in the art and technology to which these embodiments pertain will appreciate that still other alterations and changes in the described structures and methods of operation may be practiced without meaningfully departing from the principle and scope of these embodiments. Additionally, the foregoing description should not be read as pertaining only to the precise structures described and shown in the accompanying drawings, but rather should be read as consistent with and as support for the following claims, which are to have their fullest and fairest scope.

We claim:

1. A multi-chamber isolated constituent syringe for eye surgery, the syringe comprising:
a first chamber for accommodating a first constituent;
a second chamber distally adjacent the first chamber for accommodating a second constituent;
a mixing chamber distally adjacent the second chamber to receive the constituents for mixing therein;
at least one mixing mechanism of the syringe for reaching the mixing chamber; and
a needle in fluid communication with the mixing chamber for delivery of the combined mixed constituents into an eye of a patient.

2. The syringe of claim 1 wherein the chambers are isolated from one another by barriers selected from a group consisting of medical foil, a pressure degradable membrane and a rupture disk.

3. The syringe of claim 1 wherein the first chamber is a wet chamber and the first constituent is a liquid and the second chamber is a dry chamber and the second constituent is a dry constituent.

4. The syringe of claim 3 wherein the liquid constituent is about 2.00 milliliters (ml) of a polymer solution, the dry constituent is about 0.10 ml of a dry polymer reactant and the combined mixed constituents comprise a retinal patch.

5. The syringe of claim 1 wherein the mixing chamber accommodated dedicated airspace of a volume tailored to the constituents for mixing.

6. The syringe of claim 1 wherein the mixing mechanism is an auger implement coupled to a plunger for breaching the barriers with the constituents.

7. The syringe of claim 1 wherein the mixing chamber further comprises one of baffling and a piezo-electric actuator for enhancing mixing of the constituents.

8. The syringe of claim 1 further comprising a dual depressor actuator at an external location of a housing accommodating the chambers, the dual depressor actuator configured to provide simultaneous surgical control over the mixing mechanism and a plunger for directing the constituents to the mixing chamber.

9. A multi-barrel isolated constituent syringe for eye surgery, the syringe comprising:
a first chamber for accommodating a first constituent;
a second chamber parallel the first chamber for accommodating a second constituent; and
a mixing chamber distally adjacent the first and second chambers to receive the constituents therefrom for mixing therein;
at least one mixing mechanism of the syringe for reaching the mixing chamber; and
a needle in fluid communication with the mixing chamber for delivery of the combined mixed constituents into an eye of a patient.

10. The syringe of claim 9 wherein the mixing mechanism is in direct contact with a dislodgeable element to introduce the constituents to the mixing chamber at substantially the same time.

11. The syringe of claim 9 wherein the mixing mechanism is configured for reciprocating within the mixing chamber, the mixing chamber further comprising baffling to enhance the mixing during the reciprocating.

12. A method of mixing multiple constituents in a syringe for delivery into an eye of a patient, the method comprising:
breaching at least one isolating barrier to advance the constituents from isolated chambers into a mixing chamber;
mixing the constituents in the mixing chamber with a mixing mechanism; and
delivering a combined mixture of the constituents into the eye of the patient.

13. The method of claim 12 wherein the constituent chambers are one of sequential leading to the mixing chamber for introducing the constituents to the mixing chamber in a partially combined manner and parallel for simultaneous introducing of the constituents to the mixing chamber in a substantially uncombined manner.

14. The method of claim 12 further comprising filtering debris from the combined mixture within the mixing chamber during the delivering, the debris selected from a group consisting of barrier debris and unmixed constituent.

15. The method of claim 12 wherein the breaching, the mixing and the delivering are directed by a pneumatic power cell device that includes a dual depressor actuator to allow separate mixing actuation apart from the breaching and delivering.
